(19) United States
(12) Patent Application Publication
(10) Pub. No.: US 2009/0155321 A1
(43) Pub. Date: Jun. 18, 2009

(54) COMPOSITIONS WITH ENCAPSULATED COLORING AGENTS AND METHOD TO IMPART A HEALTHY SKIN APPEARANCE

(75) Inventors: Bijan Harichian, Brookfield, CT (US); Lei Huang, Orange, CT (US); Zhi-xing Jiang, Southbury, CT (US); Bivash Ranjan Dasgupta, Hamden, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., d/b/a UNILEVER, Englewood Cliffs, NJ (US)

(21) Appl. No.: 12/194,667

(22) Filed: Aug. 20, 2008

Related U.S. Application Data
(60) Provisional application No. 61/012,973, filed on Dec. 12, 2007.

(51) Int. Cl. A61K 8/02; A61Q 19/00

(52) U.S. Cl. 424/401

(57) ABSTRACT

A cosmetic composition and method of imparting a healthy appearance to skin is provided which includes using a composition formed with about 0.1 to about 20% by weight of the composition of beads, from about 1 to about 80% by weight of the beads of a first coloring agent incorporated within a matrix of the beads, and a cosmetically acceptable carrier, the composition having a hue less than 25°, the beads being coated and having an exterior color other than that of the first coloring agent.

### BACKGROUND OF THE INVENTION

1. Field of the Invention

The invention concerns cosmetics particularly moisturizers formulated with encapsulated coloring agents that deliver a healthy appearance to skin.

2. The Related Art

Skin color is a major indicia of a healthy looking appearance. Make-up formulas have been designed to mimic a person's skin tones. High loadings of colorants are optical ingredients necessary for these formulations. These loadings achieve a covering purpose at the expense of inferior tactile sensory properties.

Less loaded formulas, particularly moisturizers can provide the desired tactile sensory benefits. But these formulas do not address facial color issues.

An approach alternative to the matte effect of make-up has been the use of soft focus particles. Here the incoming light is distorted by scattering (lensing). Components of these formulas operate as lenses to bend and twist light into a variety of directions.

U.S. Pat. No. 5,997,890 (Sine et al.), U.S. Pat. No. 5,972,359 (Sine et al.), and U.S. Pat. No. 6,174,533 B1 (SaNogueira, Jr.) are all directed to topical compositions that provide good coverage of skin imperfections. The solution proposed by these documents is a soft focus effect utilizing a metal oxide with a refractive index of at least about 2 and a neat primary particle size of from about 100 to about 300 nm. Preferred particulates are titanium dioxide, zirconium oxide and zinc oxide.

A significant disadvantage of titanium dioxide and zinc oxide is the whitening effect upon the skin. An undesirable ashen appearance is unfortunately created.

U.S. Patent Application 2005/0079190 A1 (Polonka) discloses use of solid single-crystal flat platy particles which in cosmetic skin care compositions provide consumer-desired properties of the appearance of natural skin radiance. Suitable platy particles include bismuth oxy-chloride, aluminum oxide, zirconium oxide and boron nitride.

A challenge which has not been fully met by the known art is to understand the fundamental basis of a healthy skin tone enhancing rather than covering over natural skin. New approaches are necessary which not only deliver a healthy skin appearance but that also impart desirable tactile sensory benefits.

### SUMMARY OF THE INVENTION

A cosmetic composition is provided which includes from about 0.1 to about 20% by weight of the composition of beads, from about 1 to about 80% by weight of the beads of a first coloring agent incorporated within a matrix of the beads, and a cosmetically acceptable carrier, the composition having a hue of less than 25°, the beads being coated and having an exterior color other than that of the first coloring agent.

Further, there is provided a method for imparting a healthy appearance to skin which includes:
(A) providing a cosmetic composition that includes:

(i) from about 0.1 to about 20% by weight of the composition of beads having an average particle size ranging from about 100 to about 3000 micron (μm);

(ii) from about 1 to about 80% by weight of the beads of a first coloring agent incorporated within a matrix of the beads, the composition having a hue value less than 25° and being a color which is not a natural skin color;

(iii) from about 0.1 to about 10% by weight of the beads of a second coloring agent held within a coating surrounding the matrix, the coating having a color other than that of the first coloring agent;

(iv) a cosmetically acceptable carrier; and
(B) applying the cosmetic composition to human skin.

### DETAILED DESCRIPTION OF THE INVENTION

Human skin has a unique color range. This color or spectral reflectance range is largely shaped by skin chromophores such as hemoglobin and melanin. Hemoglobin is an essential component of blood and of life. Hemoglobin has a characteristic absorption spectrum in the visible light range. It absorbs strongly at blue and green wavelength and much less so at red wavelength with an absorption peak at about 550 nm. This is shown in the skin spectral reflectance as a hemoglobin dip at 550 nm. As blood is intrinsically associated with life, the presence of the hemoglobin dip has been found to be a key to the perception of healthy looking skin.

Further, we have divided the natural skin color space into four regions defined by the lightness (L*) and hue (h) values, where L* is part of the L*a*b color scale and h (in degrees)=arctan (b*/a*):

1) Light and Cool: 70>L*>55 and 55°>h>40°,

2) Light and Warm: 70>L*>55 and 70°>h>55°,

3) Dark and Cool: 55>L*>35 and 55°>h>40° and

4) Dark and Warm: 55>L*>35 and 70°>h>50°.

Most marketed make-up products belong to one of these four color spaces. The color spaces of our invented products lies outside the four regions listed above. For example, in the “Light and Cool” region, our product in the bottle occupies a color space with lightness 75>L*>55 and hue h<25°. This color space is different from the color space of traditional colored/tinted skin care products. Compositions of this invention, when applied on skin, provide a perceptible change in skin appearance.

Thus, we have found that we can deliver consumer desired skin color appearance from a non-makeup (e.g. a moisturizer) by setting product color far away from the natural skin color space. The color spaces of the inventive products have a combined color with lightness L* less than 75 and hue h less than 25. This is different from traditional colored products for skin care where product colors are close to skin colors with hue from about 30 to 70. Of particular distinction is that colorants of this invention lower the spectral reflectance at about 550 nm. This will help visualize the healthy look effect.

A variety of colorants may serve as the first coloring agent according to the present invention. These may generally be referred to as organic dyes, inorganic colorants (e.g. pigments), inorganic salts of organic dyes (e.g. Lakes) and combinations thereof. Particularly preferred are the pigment iron oxides. These often are mixtures of differently colored iron oxides which may be red, yellow, brown and black. Lakes are characterized by an organic pigment obtained by precipitation of a water-soluble dye, frequently a sulfonic acid, by an inorganic cation or an inorganic substrate such as aluminum hydrate. Typical organic acids subject to being Laked are tannic acid and inorganic heteropolyacids like phosphotungstic and phosphomolybdic acids. Examples include Methyl Violet (Pigment Violet 3), Victoria Blue (Pigment Blue 1) and Malachite Green (Pigment Green 4). Also useful are Manganese Violet, ultramarines (polysulfide sodium aluminum sulfosilicates), chromium oxides, ferric ferrocyanide and their combinations among themselves and other colorants.

Amounts of the first coloring agent, which is defined as plural including a total of all first coloring constituents, may range from about 1 to about 80%, preferably from about 5 to about 40%, optimally from about 10 to about 25% by weight of the bead. The relative weight ratio of pigment to matrix may range from about 0.8:1 to about 1:100, preferably from 0.2:1 to 1:20.

Beads of the present invention are formed of a matrix which may either constitute a natural polymer, a synthetic polymer or combinations thereof. Typical natural polymers are carboxymethyl cellulose, cellulose acetate phthalate, ethyl cellulose, propyl hydroxycellulose, gelatin, gum arabic, starch, methyl cellulose, waxes, alginates and combinations thereof. Among the synthetic polymers are included polyvinyl alcohol, polyethylene, polypropylene, polyether, ethylene vinyl acetate copolymer, polyvinylidene chloride, polyhydroxyethyl methacrylate, polyacrylate, polymethacrylate, polyacrylonitrile, polyvinylpyrrolidone, polyvinyl acetate and combinations thereof. These polymers may serve as a matrix for the beads and can be surrounded by a separate encapsulating coating. Alternatively, the polymers can serve as a shell and thereby be a coating around the first coloring agent.

Beads of this invention preferably will have matrices based on the natural polymers of cellulose and cellulose derivatives. Besides cellulose, the derivatives may include hydroxypropyl methyl cellulose, hydroxyethyl cellulose, hydroxypropyl cellulose, carboxymethyl cellulose and combinations thereof. Sugars may also be employed as a component of the matrices. These include mannitol, sorbitol, xylitol and mixtures thereof. Relative amounts of sugar to cellulose (and/or cellulose derivative) range from about 3:1 to about 1:3, preferably from about 2:1 to about 1:2 by weight of the matrix. Most preferred is a matrix of mannitol, cellulose and hydroxypropyl methyl cellulose. Amounts of hydroxypropyl methyl cellulose may range from about 0.01 to less than about 1% by weight of the matrix. Commercially this matrix in the form of beads is available from Induchem USA, Inc. under the tradename Induchem Unispheres. These beads are formed from a homogeneous spherical semi-solid matrix core consisting of mannitol, cellulose and hydroxypropyl methyl cellulose. An outer coating surrounds the matrix to insure coloring agent is prevented from leaving. This coating contains a plasticizer, polymer and a colorant.

Advantageously beads of this invention should be swellable from water when in contact with an aqueous system. Swelling may increase the volume of the beads by an amount from about 5% to about 20%, preferably from about 10% to about 18%, as measured at 25° C. over a 20 day period using 0.2% by weight of beads in water. Amount of swelling when greater than the aforedescribed range results in leakage of coloring agent from the matrix of the beads. Equally undesirable is minor or non-swelling which results in beads that do not release coloring agent during rub-in of composition onto the skin. Too much or too little swelling is therefore disadvantageous for purposes of this invention. A hydrophilic matrix functioning as a sponge with defined uptake of water is a desirable feature of this invention. Coloring agents inside the beads should be easily broken allowing them to spread during rub-in process onto skin.

The first coloring agent is embedded within the matrix of easily frangible beads. A polymeric coating surrounds each of the beads to prevent their premature dissolution. The beads in an aqueous medium can swell but no leakage of coloring agent (and thereby of color) will occur because of coloring agent insolubility.

A second coloring agent may be held within a coating surrounding the matrix. This agent will have a color other than that of the first coloring agent. Most suitable as a second coloring agent are white colors. This may be imparted by titanium dioxide, calcium carbonate, calcium silicate, clay, barium sulfate, zinc oxide, mica, alumina, magnesium carbonate, hydroxyapatite and mixtures thereof. Amounts may range from about 0.01 to about 10%, preferably from about 0.1 to about 5% by weight of the beads of the second coloring agent.

Amounts of the beads may range from about 0. 1 to about 20%, preferably from about 0.5 to about 15%, optimally from about 2 to about 10% by weight of the cosmetic composition.

Average particle size of beads according to the present may range from about 100 to about 3,000 micron (μm), preferably from about 500 to about 1,300 micron (μm), optimally from about 700 to about 900 micron (μm).

As used herein, the term “cosmetic composition” is intended to describe compositions for topical application to human skin, including leave-on and wash-off products. Preferably the term encompasses a fluid liquid, and most particularly a moisturizer rather than a make-up product.

The term “skin” as used herein includes the skin on the face, neck, chest, back, torso, arms, axillae, hands, legs, and scalp.

As used herein, “color” is a general term intended to cover human perception of color and includes variations in lightness/darkness and/or variations in hue.

Lightness is defined in terms of the L* parameter in the L*-a*-b* color space, which will be discussed in more detail hereinbelow. The greater the L* value, the lighter the skin. The smaller the L* value, the darker the skin, indicating higher melanin content.

Hue is defined as the color component on a red to yellow spectrum. More specifically, hue is defined in terms of the a* and b* parameters in L*-a*-b* color space, as follows:

Hue=tan⁻¹(b*/a*)

Usually for skin color, a* and b* are greater than zero, so the smaller the Hue value, the more red the color.

This color system is known as the Commission Internationale de l'Eclairage (CIE) L*a*b* color system, where:

L*=Black to white (luminance) from 0 to 100 [L*=0 represents Black]

a*=green to red from −60 to +60

b*—blue to yellow from −60 to +60

as measured by a chromameter, such as for example a hand held Minolta CM2002 chromameter.

Compositions of the present invention will also include a cosmetically acceptable carrier. Water is the most preferred carrier. Amounts of water may range from about 1 to about 99%, preferably from about 5 to about 90%, more preferably from about 35 to about 70%, optimally between about 40 and about 60% by weight. Ordinarily the compositions will be water and oil emulsions, most preferably of the oil-in-water variety. Indeed, in certain instances the water-in-oil emulsions should be avoided because the hydrophobicity will inhibit water dissolution of the bead matrix (i.e. cellulose) to release pigment upon rub-in of the composition onto skin.

Other cosmetically acceptable carriers may include mineral oils, silicone oils, synthetic or natural esters, fatty acids and alcohols and humectants. Amounts of these materials may range from about 0.1 to about 50%, preferably from about 0.1 to about 30%, more preferably from about 1 to about 20% by weight of the composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5; silicon atoms.

Linear volatile silicone materials generally have viscosities less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C.

Among suitable esters are:

(1) Alkenyl or alkyl esters of fatty acids having 10 to 20 carbon atoms. Examples thereof include isopropyl palmitate, isopropyl isostearate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxy-ethylene sorbitan fatty acid esters are satisfactory polyhydric alcohol esters.

(4) Wax esters such as beeswax, spermaceti, myristyl myristate, stearyl stearate.

(5) Sterols esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include glycerol (also known as glycerin), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerin. The amount of humectant may range anywhere from 0.5 to 30%, preferably between 1 and 15% by weight of the composition.

Emulsifiers may be present in cosmetic compositions of the present invention. Total concentration of the emulsifier may range from about 0.1 to about 40%, preferably from about 1 to about ²⁰%, optimally from about 1 to about 5% by weight of the total composition. The emulsifier may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Thickening agents may be included in compositions of the present invention. Particularly useful are the polysaccharides. Examples include starches, natural/synthetic gums and cellulosics. Representative of the starches are chemically modified starches such as aluminum starch octenylsuccinate. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are still a further class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature of Sodium Acrylate/Sodium Acryloyldimethyl Taurate and Acryloyl Dimethyltaurate/Vinyl Pyrrolidone Copolymer.

Amounts of the thickener may range from about 0.001 to about 5%, preferably from about 0.1 to about 2%, optimally from about 0.2 to about 0.5% by weight.

Fragrances and abrasives may also be included in compositions of the present invention. Each of these substances may range from about 0.05 to about 5%, preferably between 0.1 and 3% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

It should be noted that in specifying any range of concentration or amount, any particular upper concentration can be associated with any particular lower concentration or amount.

The following Examples will more fully illustrate the embodiments of this invention. All parts, percentages and proportions referred to herein and in the appended claims are by weight unless otherwise indicated.

### EXAMPLE 1

A series of lotions according to the present invention are reported in the Table below.

TABLE I

| | Sample (Weight %) | | | | | |
|---|---|---|---|---|---|---|
| Component | A | B | C | D | E | F |
| Stearic Acid | 2.40 | 2.40 | 3.00 | 3.00 | 1.95 | 1.95 |
| Glyceryl | 1.40 | 1.50 | 1.50 | 1.05 | 1.05 | 3.10 |
| Monostearate/Stearamide AMP | | | | | | |
| Glycerol Monostearate | 0.65 | 0.65 | 0.65 | 0.65 | 0.65 | 0.65 |
| Cetyl Alcohol | 0.37 | 0.37 | 0.37 | 0.37 | 0.37 | 0.37 |
| Petrolatum | 1.25 | 2.25 | 3.59 | 0.80 | 0.80 | 4.35 |
| Isopropylmyristate | 1.30 | 1.30 | 1.30 | 1.30 | 1.30 | 1.30 |
| Disodium EDTA | 0.05 | 0.05 | 0.05 | 0.05 | 0.05 | 0.05 |
| Induchem Beads* | 2.00 | 1.00 | 4.00 | 4.00 | 8.00 | 8.00 |
| Glycerin | 10.00 | 10.00 | 5.00 | 5.00 | 5.00 | 10.00 |
| Simulgel EG ® | 0.75 | 0.75 | 0.75 | 0.75 | 0.75 | 0.75 |
| Titanium Dioxide | 0.10 | 0.20 | 0.80 | 0.10 | 0.20 | 0.10 |
| Triethanolamine (99%) | 0.70 | 0.70 | 0.90 | 0.90 | 0.60 | 0.60 |
| Glydant Plus ® | 0.09 | 0.09 | 0.09 | 0.09 | 0.09 | 0.09 |
| DMDM Hydantoin | 0.17 | 0.17 | 0.17 | 0.17 | 0.17 | 0.17 |
| Silicone 50 ct | 1.50 | 1.50 | 1.50 | 1.50 | 1.50 | 1.50 |
| Silicone DC 1501 ® | 0.50 | 0.50 | 0.50 | 0.50 | 0.50 | 0.50 |
| Fragrance | 0.30 | 0.30 | 0.30 | 0.30 | 0.30 | 0.30 |
| Water | Balance | Balance | Balance | Balance | Balance | Balance |

*Beads are provided with a mixture of variously colored iron oxides and also titanium dioxide, the combination of which is not a natural skin color.

The samples of Table 1 are formulated in the following manner. A reactor is charged with the deionized water and disodium EDTA. Heat is applied till 60° C. in combination with stirred mixing. The Induchem beads are added and heating continued for 10 minutes. Simulgel EG® is added to the reactor and the temperature maintained at 77-80° C. for 10 to 15 minutes. In a separate vessel, the oil phase components are added. Light mixing of the batch is performed with heating in a water bath to 75-77° C. The water reactor is maintained at 60-65° C. and glycerin, titanium dioxide and triethanolamine are slowly charged to the reactor. Continuous mixing is done until the aqueous system is uniform. Very slowly the oil phase is added to the water phase at 75-77° C. under moderate mixing. After full emulsification, the batch is agitated for a further 5 minutes. Thereupon the resultant emulsion is homogenized using an ARDE Barenco® apparatus for 20-30 seconds at 35%. The resultant system is then topped with further deionized water. Cooling is then begun with a large sweep (50 rpm) mixer. Preservatives Glydant Plus® and DMDM Hydantoin are then added with the batch held at 50-55° C. At a temperature of 45-50° C., the fragrance is charged to the reactor. Heating is then discontinued and mixing stopped when the temperature reaches 38-40° C.

### EXAMPLE 2

A series of experiments were conducted to compare different types of beads for purposes of this invention. Focus was on the ability of a bead to hide first coloring agents within a matrix of the bead. These beads need to be relatively unnoticeable within a moisturizer composition but upon being rubbed into skin must deliver color effects. The following four types of beads were evaluated.

(1) Wax

A wax was heated to a melt. Therein was distributed 0.2% of DC Red 17. Cooling resulted in solid wax beads of red color thereby forming a bead matrix. The matrix was then added into a second portion of melted wax, and the combination quickly emulsified in a water phase. In this process, the emulsion formation was controlled so that the color bearing matrix only partially melted allowing the non-colored wax second portion to coat the bead. The result was a final bead with an exterior slightly pink in appearance surrounding a strong red colored matrix.

(2) Calcium Silicate

Red iron oxide (0.2%) was encapsulated through a water-oil-water triplex emulsion process. Water dispersible iron oxide was placed in the inner water phase which was a sodium silicate solution. Calcium chloride was placed in the outer water phase. Upon mixing, the calcium from the outer water phase interacted with the silicate of the inner water phase thereby forming a calcium silicate shell around the iron oxide. The resultant beads were essentially white in exterior color.

(3) Polyamide

Beads were prepared by dispersing 0.2% DC Red 17 and either adipic acid or terephthaloyl chloride in an oil phase of an emulsion. Di-amine and tri-amine mixtures were placed in the water phase. Polyamide formed at the oil/water emulsion interface to encapsulate colorant within the oil phase. The result were beads of slightly pinkish exterior color encapsulating a relatively strong red colorant.

(4) Hydrophilic Polysaccharide

These beads were formed from a homogeneous spherical semi-solid matrix consisting of mannitol (25-50%), cellulose (15-30%) and hydroxypropyl cellulose (less than 1%). A combination of titanium dioxide (0.1-10%) and red iron oxide (5-39%) were included to give a final loading of approximately 40% total pigment. This red matrix was then surrounded by an outer coating formed of an acrylate polymer (1-5%), a nonionic alkoxylated dispersant (5-10%) and whitening agent (2-10%). The resultant beads were visually white. These beads can be sourced from Induchem USA, Inc. under the tradename of Unispheres.

Each of the four types of beads were.evaluated for their performance. See Table II.

TABLE II

| | | Rub-In |
|---|---|---|
| | Color Hiding | Color |
| Bead Type | Efficiency | Transfer |
| (1) Wax | Poor | Fair |
| (2) Calcium Silicate | Good | Good |
| (3) Polyamide | Poor | Fair |
| (4) Hydrophilic Polysaccharide | Excellent | Excellent |

Wax (1) and polyamide (3) bead technology was inadequate to hide the bright red colors of the dye/pigments encapsulated therein. The calcium silicate (2) efficiently masked the red color. Size, shape and hardness of the capsules were, however, difficult to control. The best among the encapsulating systems was the hydrophilic polysaccharide. These beads were highly efficient at hiding the red color and very effective at releasing this color when the lotion in which they were suspended was rubbed into skin.

What is claimed is:

1. A cosmetic composition comprising from about 0.1 to about 20% by weight of the composition of beads, and from about 1 to about 80% by weight of the beads of a first coloring agent incorporated within a matrix of the beads, and a cosmetically acceptable carrier, the composition having a hue less than 25°, the beads being coated and having an exterior color other than that of the first coloring agent.

2. The composition according to claim 1 wherein the beads have an average particle size ranging from about 100 to about 3,000 micron (μm).

3. The composition according to claim 1 wherein the beads have an average particle size ranging from about 500 to about 1,300 micron (μm).

4. The composition according to claim 1 further comprising from about 0.1 to about 10% by weight of the beads of a second coloring agent held within a coating surrounding the matrix, the second coloring agent is white and the first coloring agent is other than white.

5. The composition according to claim 1 wherein the matrix comprises a sugar and a cellulose or a cellulose derivative.

6. The composition according to claim 1 wherein the matrix comprises mannitol and cellulose.

7. The composition according to claim 1 wherein the hue is not a natural skin color.

8. The composition according to claim 6 wherein the mannitol and cellulose are present in a relative ratio ranging from about 3:1 to about 1:3.

9. The composition according to claim 1 wherein the beads are swellable in water by an amount from about 5% to about 20% by volume of the beads.

10. A method for imparting a healthy appearance to skin comprising:
(A) providing a cosmetic composition, the composition comprising:
(i) from about 0.1 to about 20% by weight of the composition of beads having an average particle size ranging from about 100 to about 3000 micron (μm);
(ii) from about 1 to about 80% by weight of the beads of a first coloring agent incorporated within a matrix of the beads, the composition having a hue value less than 25° and being a color which is not a natural skin color;
(iii) from about 0.1 to about 10% by weight of the beads of a second coloring agent held within a coating surrounding the matrix, the coating having an exterior color other than that of the first coloring agent;
(iv) a cosmetically acceptable carrier; and
(B) applying the cosmetic composition to human skin.
